(19) United States
(12) Patent Application Publication
(10) Pub. No.: US 2011/0106017 A1
(43) Pub. Date: May 5, 2011

(54) APPARATUS AND METHOD FOR IMPROVING ACCURACY AND EFFICIENCY FOR SYRINGES

(76) Inventors: JAMES W. BAKER, Lake Oswego, OR (US); Gary A. Kirian, Lake Oswego, OR (US); Luke Barratt, Portland, OR (US)

(21) Appl. No.: 12/611,029

(22) Filed: Nov. 2, 2009

(51) Int. Cl. A61M 5/315; G01N 1/14; B67D 7/08

(52) U.S. Cl. 604/208; 73/864.11; 222/1; 222/386

(57) ABSTRACT

Embodiments relate to a method and system for controlling a volume of liquid to be drawn and metered. One embodiment relates to a restraining divider for a syringe for controlling a volume of liquid drawn and metered by the syringe comprising a grooved side with a first groove and a second groove. Either the first groove or the second groove fits a shaft of a plunger of the syringe, wherein the length of at least one of the first groove, the second groove or the groove side provides a gap between a compressed thumb flange of said syringe and the body of the syringe.

### FIELD OF THE INVENTION

The invention relates to systems and methods for controlling a volume of liquid to be dispensed. More particularly, embodiments relate to systems and methods for controlling a liquid to be drawn and metered by a syringe.

### BACKGROUND OF THE INVENTION

Syringes are essential tools used in a wide range of fields. Syringes serve medical purposes such as delivering or dispensing medication to patients. Syringes are also frequently used in laboratories for various purposes such as for dispensing specific amounts of a liquid into a solution.

Because syringes are sometimes needed to be drawn and metered with a specific volume of liquid, accuracy and precision are both desired in the drawing and metering process. In the laboratory, even a slight miscalculation of a measurement can result in a costly error. Time can also be a pressure on the drawing and metering process. In these situations, the ability to draw and meter using the syringe quickly is very beneficial.

In many instances, users depend on their eye sight when measuring the amount of liquid drawn by the syringe. This manual process leaves significant room for human error and requires adequate light and excellent eye sight. Measuring via human eyes requires a certain amount of time to take in an accurate measurement. This amount of time will vary depending on the experience and skill of the user as well as the accuracy desired. What is needed, therefore, is an easy, repeatable, verifiable way for users to quickly and accurately draw and meter specific volumes of liquid into syringes.

### SUMMARY OF THE INVENTION

One aspect of the present invention is an apparatus including a restraining divider for a syringe for controlling a volume of liquid drawn and metered by the syringe. The restraining divider comprises a grooved side with a first groove and a parallel second groove, wherein either the first groove or the second groove fits a shaft of a plunger of the syringe. The dimension (length for example) of at least one of the first groove, the second groove or the groove side provides a gap between a compressed thumb flange of the syringe and the body of the syringe. The gap is a specific distance intended to correspond with a specific volume of liquid to be drawn and metered by the syringe.

In another aspect of the present invention, the restraining divider may include a non-grooved side. The non-grooved side can have an aperture, wherein the aperture provides a means to connect the restraining divider to a key chain or key ring.

Another aspect of the invention provides a method of drawing and metering liquid from a syringe. In this method, a divider is inserted on a shaft of a plunger of the syringe between a thumb flange and a body of the syringe. The thumb flange is compressed against the divider so that there is no space between the thumb flange and the divider. The divider is then removed from the shaft of the plunger of the syringe. The syringe is able to meter a volume of liquid corresponding to a distance pressed between the thumb flange and the body of the syringe which is equal to the width of the divider or length of the groove.

Still another embodiment relates to a system for metering and drawing a specific volume of liquid, the system comprises a syringe having a thumb flange and a plunger having a shaft and a restraining divider. The divider comprises a main body having opposing first and second ends; at least one groove defined by the main body proximate one of the first and second ends, the groove having a predefined length (L) and width (W), where the grove width (W) is sized to fit the plunger shaft, such that the plunger shaft may be removably seated in the groove, and at least groove length (L) is sized to determine the specific amount of liquid to be drawn and metered.

The foregoing and other features and advantages of the invention will become further apparent from the following detailed description of the presently preferred embodiment, read in conjunction with the accompanying drawings. The drawings are not to scale. The detailed description and drawings are merely illustrative of the invention rather than limiting, the scope of the invention being defined by the appended claims and equivalents thereof.

### BRIEF DESCRIPTION OF THE DRAWINGS

The present invention is illustrated by the accompanying drawings of various embodiments and the detailed description given below. The drawings should not be taken to limit the invention to the specific embodiments but are for explanation and clarity. The detailed description and drawings are merely illustrative of the invention rather than limiting, the scope of the invention being defined by the appended claims and equivalents thereof. The foregoing aspects and other attendant advantages of the present invention will become more readily appreciated by the detailed description taken in conjunction with the accompanying drawings. Throughout the various figures, like reference numbers refer to like elements.

FIG. 1 is a perspective view of one embodiment of the present invention.

FIG. 2 is another perspective view of one embodiment of the present invention.

FIG. 3 is a side flat view of one embodiment of the present invention.

FIG. 4 is a side view of one embodiment of the present invention.

FIG. 5 is a flow chart of one embodiment of a method of using the apparatus of the present invention.

FIG. 6 is another perspective view of one embodiment of the present invention.

### DETAILED DESCRIPTION OF PRESENTLY PREFERRED EMBODIMENTS

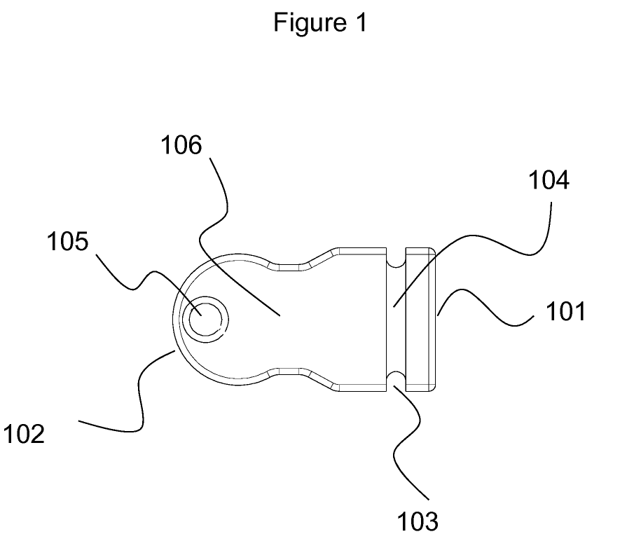

FIG. 1 illustrates one embodiment of the present invention. The apparatus has two sides or surfaces proximate a grooved side (101) and an opposing non-grooved side (102). The grooved side is proximate the side that is proximate the groove (103). There is a parallel groove on the other or opposing side of the apparatus separated by a slot (104). However, it is also contemplated that the grooves may be offset from each other. The groove (103) fits a shaft of a plunger of a syringe. The shaft of the plunger can rest, be seated, or be releasably contained within the groove (103) of the apparatus. It is contemplated that the apparatus may be comprised of a material including plastic, polymers, glass, ceramic, photo luminescent (Glow-in-the-Dark) materials which includes Strontium Aluminates, Zinc Sulphide and the like or combinations thereof and may be used in well light rooms or in rooms having low light levels.

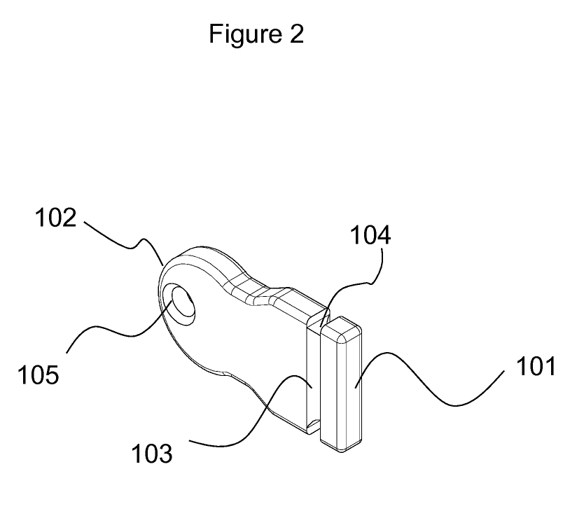

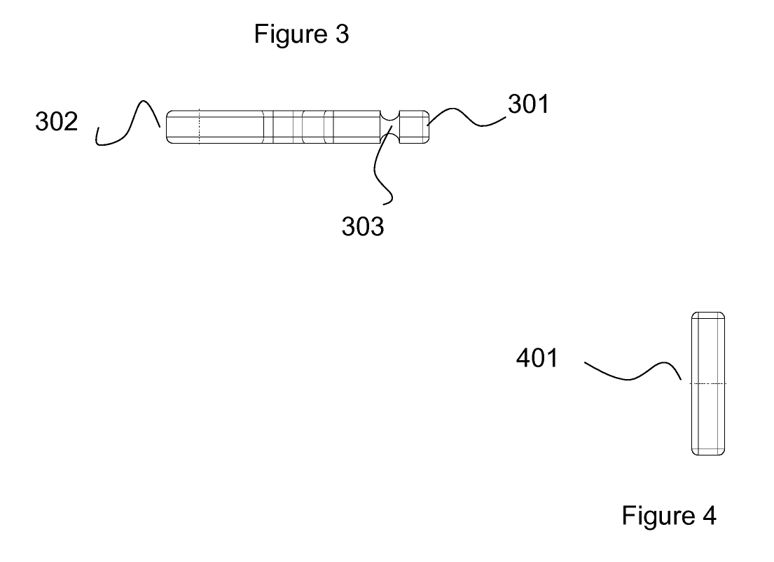

FIG. 2 illustrates a preferred embodiment from a different perspective. The apparatus has a grooved side (101) and a non-grooved side (102). FIG. 2 shows one of the parallel grooves (103) which is separated from the other groove by a slot (104).

A thumb flange of the syringe is compressed so that the apparatus is snug between the thumb flange and a body of the syringe. The circumference of the shaft of the syringe should be equivalent to the opening of the groove (103) in order that the groove can releasably hold the shaft. If the shaft is not round, then the perimeter of the shaft should be equivalent to opening of the groove (103) in order that the groove can hold the shaft.

When the syringe draws a liquid, the syringe will only dispense up to the distance allowed by the apparatus, which is the length of the groove (103) and grooved side (101). FIG. 4 illustrates a side view of the apparatus, and shows the distance (401) that the apparatus will allow the syringe to be compressed.

The apparatus can come in different sizes. The distance or length (L) of the groove (103) or the grooved side (101, 401) corresponds to a specific volume and/or density of the liquid to be metered and dispensed. This amount may be labeled on the apparatus (106) so that the user knows which sized apparatus to use in a given situation by indicia marked thereon. In at least one embodiment, the apparatus may include grooves (103) having different lengths (L) or widths (W), enabling one apparatus to accommodate different sized syringes or dispense different volumes of liquid.

The most common desired volumes for metering and drawing are 0.05 mL, 0.1 mL, 0.15 mL, 0.2 mL, 0.25 mL, 0.3 mL, 0.5 mL, 0.5 mL and 1.0 mL. In one embodiment of the invention, the apparatuses are designed to meter these values which were calculated by compensating for the density (ρ) of water at standard conditions and inserting them into the equation 1:

Mass+0.2279=0.4370(Length)

And solving for the length of groove (103) of the apparatus. Given that:

Mass=Density*Volume

pH₂0=0.9965 g/cm̂3

The following equation as an example was used to solve for the length of the groove of the apparatus in order to meter 0.25 mL.

Length=2.288(Density*Volume)+0.5215

Length=2.288*0.9965 g/cm̂3*0.25 cm̂3+0.5215

Length=1.091498 in

This operation was conducted across a range of values between 0.05 mL and 1.0 mL as depicted in Table 1.

TABLE 1
TOTAL VOLUMETRIC RELATIONSHIP

| ρ = 0.9965 g/cm\hat{ }3 | | |
|---|---|---|
| Volume (cc) | Divider length (in.) | Mass (g) |
| 0.05 | 0.6339 | 0.0498 |
| 0.1 | 0.7481 | 0.0997 |
| 0.15 | 0.8622 | 0.1495 |
| 0.2 | 0.9764 | 0.1993 |
| 0.25 | 1.0905 | 0.2491 |
| 0.3 | 1.2046 | 0.2990 |
| 0.35 | 1.3188 | 0.3488 |
| 0.4 | 1.4329 | 0.3986 |
| 0.45 | 1.5471 | 0.4484 |
| 0.5 | 1.6612 | 0.4983 |
| 0.55 | 1.7753 | 0.5481 |
| 0.6 | 1.8895 | 0.5979 |
| 0.65 | 2.0036 | 0.6477 |
| 0.7 | 2.1178 | 0.6976 |
| 0.75 | 2.2319 | 0.7474 |
| 0.8 | 2.3460 | 0.7972 |
| 0.85 | 2.4602 | 0.8470 |
| 0.9 | 2.5743 | 0.8969 |
| 0.95 | 2.6885 | 0.9467 |
| 1 | 2.8026 | 0.9965 |

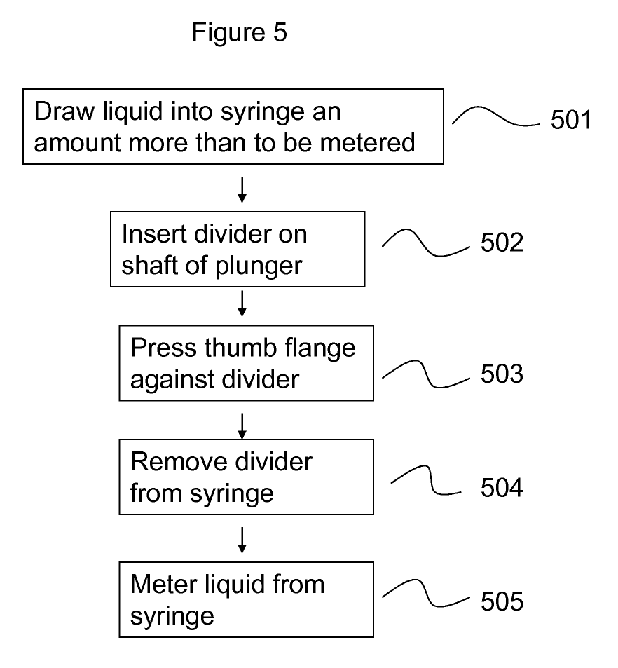

FIG. 5 depicts a flow chart which shows a method of using one embodiment of the divider of the invention. A syringe draws liquid from a source an amount greater than the volume required (block 501). A divider is inserted on a shaft of a plunger of the syringe between a thumb flange and a body of the syringe (block 502). The thumb flange is compressed against the divider so that there is no space between the thumb flange and the divider (block 503). The divider is then removed from the shaft of the plunger of the syringe (block 504). The syringe meters a volume of liquid corresponding to a distance pressed between the thumb flange and the body of the syringe which is equal to the width of the divider or length of the groove into a selected destination with great accuracy (block 505).

As shown in FIG. 1 and FIG. 2, the apparatus can also optionally include an aperture (105). This aperture can serve a purpose such as providing a means to be attached to a chain, key ring or key chain. Several apparatuses of the invention customized for different volumes can be stored together on a single key ring or key chain in order to improve the storage and accessibility of the invention.

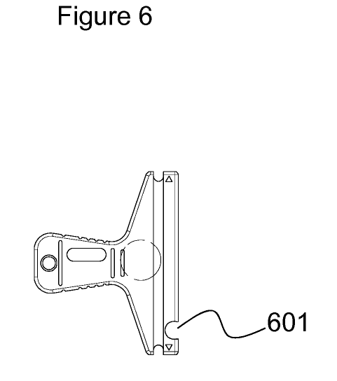

FIG. 6 illustrates another embodiment of the invention. The invention may also contain a slot (601) used as a “go no-go” gauge that will pass over the barrel diameter of the design prescribed syringe but will not pass over a larger barrel diameter non-prescribed syringe. The “go no-go” gauge (601) can serve as a safety precaution to ensure that syringes only of the prescribed size or smaller are used.

The disclosed invention alleviates many of the concerns faced in a laboratory environment. The amount of re-experimenting due to incorrect measurements will be reduced by consistently obtaining more accurate measurements. The accurate measurements can also be made at a significantly quicker speed then known manual methods, thus improving the efficiency for the users.

Any mechanical precision dispensing of fluid by hand with a syringe can benefit by use of the invention. One example might be injections given in an ambulance in route to a care facility ground or life flight. It is contemplated that a veterinarian may draw and lock a dose with the invention by carrying the syringe with the divider in place through an animal holding enclosure at arms length. The invention is also applicable for rapid metering of dose amounts during mixing operations and mass vaccine injections during pandemics.

The invention also has tremendous benefits in the field of military combat. Users would be able to administer appropriate amounts of medication in the dark under difficult conditions. These conditions may also occur in the emergency room and EMT field use during low light or adverse weather conditions. The invention can be an aid to hospital shot nurses for improving accuracy of doses, patient and operator safety, speed of administration, and comfort and confidence of administration. The invention could also be useful for blind users where braille could be used to label the different volumes for different dividers.

The invention has been described in connection with a preferred embodiment and it is to be understood that numerous and extensive departures and modifications may be made without however department from the scope of the invention which is intended to be limited only by the scope of the appended claims. The scope of the invention is indicated in the appended claims, and all changes that come within the meaning and range of equivalents are intended to be embraced therein.

1. A restraining divider for a syringe for controlling a volume of liquid drawn and metered by the syringe comprising:
a grooved side with a first groove and a parallel second groove;
wherein either said first groove or said second groove fits a shaft of a plunger of said syringe; and
wherein a dimension of one of said first groove, said second groove or said groove side provides a gap between a compressed thumb flange of said syringe and the body of the syringe.

2. The restraining divider of claim 1 wherein said gap is a specific distance that corresponds to a desired volume of liquid to be drawn or metered.

3. The restraining divider of claim 1 further comprising a non-grooved side defining an aperture, wherein said aperture provides a means to be connected to a chain, key chain or key ring.

4. The restraining divider of claim 1 wherein said first or second grooves seat, rest, hold or releasably contain said shaft of said plunger.

5. The restraining divider of claim 4 wherein a circumference or perimeter of said shaft of said plunger matches openings defined by said first and second grooves.

6. The restraining divider of claim 1, further comprising indicia indicating volume to be measured.

7. A method for controlling a volume of liquid to be drawn and metered using a syringe comprising:
drawing the liquid into the syringe where the amount of liquid drawn into the syringe is more than the amount of liquid desired to be dispensed;
inserting a divider on a shaft of a plunger of the syringe between a thumb flange and a body of the syringe;
compressing the thumb flange against the divider so that there is no space between the thumb flange and the divider, resulting in a metered volume of the liquid able to be dispensed; and
removing said divider from the shaft of the plunger of the syringe.

8. The method of claim 7 wherein a distance pressed between said thumb flange and said body is equal to the width of the divider.

9. The method claim 8 further comprising dispensing a metered volume of liquid from the solution, wherein the distance pressed is equivalent to the metered volume.

10. The method of claim 7 wherein the divider comprises a first groove and a second parallel groove, wherein said shaft of said plunger can rest, fit or connect into either of said first or second grooves.

11. The method of claim 10 wherein said plunger has a circumference or perimeter that matches openings defined by said grooves.

12. The method of claim 11 further comprising storing and utilizing multiple dividers of specific widths in order to draw and meter different volumes of liquid as desired.

13. The method of claim 11 wherein a length (L) of the first or second grooves is related to at least the volume of the liquid to be metered and dispensed.

14. A system for metering and drawing a specific volume of liquid, the system comprising:
a syringe having a thumb flange and a plunger having a shaft; and
a restraining divider, the divider comprising:
a main body having opposing first and second ends;
at least one groove defined by the main body proximate one of the first and second ends, the groove having a predefined length (L) and width (W), where the grove width (W) is sized to fit the plunger shaft, such that the plunger shaft may be removably seated in the groove, and at least groove length L is sized to determine the specific amount of liquid to be drawn and metered.

15. The system of claim 14 wherein the main body further comprises opposing surfaces separated by an edge, each opposing surface defining at least one groove.

16. The system of claim 15 wherein the main body further comprises the edge defining opposing slots, each slot proximate the at least one groove.

17. The system of claim 14 wherein the groove length (L) is related to at least the volume of the liquid to be metered and dispensed.

18. The system of claim 17 wherein the groove length (L)=2.288*0.9965 g/cm̂3*V+0.5215, wherein V is equal to a desired volume.

19. The system of claim 14 wherein at least one surface has indicia thereon.

20. The system of claim 14 wherein the divider defines at least one aperture at one of the first and second ends.
